(19) United States
(12) Patent Application Publication
(10) Pub. No.: US 2025/0084484 A1
(43) Pub. Date: Mar. 13, 2025

(54) METHODS AND COMPOSITIONS FOR TRANSCRIPTOME ANALYSIS

(71) Applicant: Jumpcode Genomics, Inc., San Diego, CA (US)

(72) Inventor: Keith BROWN, Carlsbad, CA (US)

(21) Appl. No.: 18/728,252

(22) PCT Filed: Jan. 10, 2023
(86) PCT No.: PCT/US2023/060432

§ 371 Date: Jul. 11, 2024

Related U.S. Application Data
(60) Provisional application No. 63/298,866, filed on Jan. 12, 2022.

(51) Int. Cl. C12Q 1/6886; C12Q 1/6855

(52) U.S. Cl. CPC C12Q 1/6886; C12Q 1/6855; C12Q 2600/158

(57) ABSTRACT

Provided herein are methods and compositions for analysis of a transcriptome, for example detection of a RNA isoform, of a cell, tissue, organ, or subject.

### CROSS-REFERENCE

This application claims the benefit of U.S. Provisional Application No. 63/298,866, filed Jan. 12, 2022, which is incorporated herein by reference in its entirety.

### BACKGROUND

There are over 300,000 documented proteins but only 22,000 annotated protein coding genes in the human genome. Splice variation, exon exclusion/inclusion, alternative splice donor/acceptor sites, alternative transcriptional start sites and alternative poly A ends or UTRs likely account for much of this diversity in post transcriptional modifications of mRNA in humans. Analysis of the transcriptome may allow a better understanding of differences in mRNA transcription, splicing, and modifications leading to diverse protein products.

### SUMMARY

In an aspect, provided herein, are methods of detecting an RNA isoform. Such methods can comprise contacting a plurality of RNA molecules to a oligo(dT) primer and a reverse transcriptase to produce a plurality of cDNA molecules. Next, the method can comprise attaching adaptors to a 5′ end and a 3′ end of each of the plurality of cDNA molecules. Then, the method can comprise amplifying the plurality of cDNA molecules with a first primer and a second primer that bind to the adaptors to produce a plurality of amplification products, wherein the first primer or the second primer comprises a detectable label. Next, the method can comprise capturing at least a portion of the plurality of amplification products using an agent that binds to the detectable label. Then, the method can comprise contacting at least a portion of the plurality of amplification products to a guide RNA directed endonuclease and at least one guide RNA that specifically binds to a 5′ end or a 3′ end of at least a subset of the plurality of amplification products, thereby cleaving a 5′ end or a 3′ end of the plurality of amplification products to produce a plurality of cleaved amplification products. Then the method can comprise isolating the plurality of cleaved amplification products, thereby detecting the RNA isoform. In some cases, attaching comprises ligating. Alternatively, attaching occurs concurrently with (a) wherein the oligo(dT) primer comprises the adaptor. In some cases, the first primer binds to the adaptor at the 5′ end of each of the plurality of cDNA molecules and the second primer binds to the adaptor at the 3′ end of each of the plurality of cDNA molecules. In some cases, the first primer comprises the detectable label and the guide RNA binds to the 5′ end of the subset of the plurality of amplification products. In some cases, the second primer comprises the detectable label and the guide RNA binds to the 3′ end of the subset of the plurality of amplification products. In some cases, the method further comprises dividing the plurality of cDNA molecules to at least a first reaction volume and a second reaction volume. In some cases, the first reaction volume the first primer comprises the detectable label and the guide RNA binds to the 5′ end of the plurality of amplification products. In some cases, the second reaction volume the second primer comprises the detectable label and the guide RNA binds to the 3′ end of the plurality of amplification products. In some cases, the detectable label comprises biotin. In some cases, the agent comprises streptavidin. In some cases, the at least one guide RNA binds specifically to a 5′ end or a 3′ end of the RNA isoform. In some cases, the at least one guide RNA is provided in an amount relative to an expected amount of the RNA isoform. In some cases, the method further comprises sequencing the plurality of cleaved amplification products.

In another aspect, there are provided methods of detecting an RNA isoform comprising: contacting a plurality of RNA molecules to a oligo(dT) primer and a reverse transcriptase to produce a plurality of cDNA molecules; attaching adaptors to a 5′ end and a 3′ end of each of the plurality of cDNA molecules; amplifying the plurality of cDNA molecules with a first primer and a second primer that bind to the adaptors to produce a plurality of amplification products; contacting at least a portion of the plurality of amplification products to a cleavage deficient guide RNA directed endonuclease and at least one guide RNA that specifically binds to a 5′ end or a 3′ end of at least a subset of the plurality of amplification products; and capturing at least a portion of the plurality of amplification products using an agent that binds to the cleavage deficient guide RNA directed endonuclease, thereby detecting the RNA isoform. In some cases, attaching comprises ligating. In some cases, attaching occurs concurrently with contacting wherein the oligo(dT) primer comprises the adaptor. In some cases, the at least one guide RNA binds specifically to a 5′ end or a 3′ end of the RNA isoform. In some cases, the at least one guide RNA is provided in an amount relative to an expected amount of the RNA isoform. In some cases, the method further comprises sequencing the plurality of captured amplification products.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows a schematic of alternative mRNAs resulting from a single transcription product.

FIG. 2A shows a reverse transcription step in a method of RNA transcriptome analysis.

FIG. 2B shows a template switch step in a method of transcriptome analysis.

FIG. 2C shows a PCR step in a method of transcriptome analysis.

FIG. 2D shows a capture step in a method of transcriptome analysis.

FIG. 2E shows a CRISPR cleavage step in a method of transcriptome analysis.

FIG. 3 shows an alternative method of transcriptome analysis.

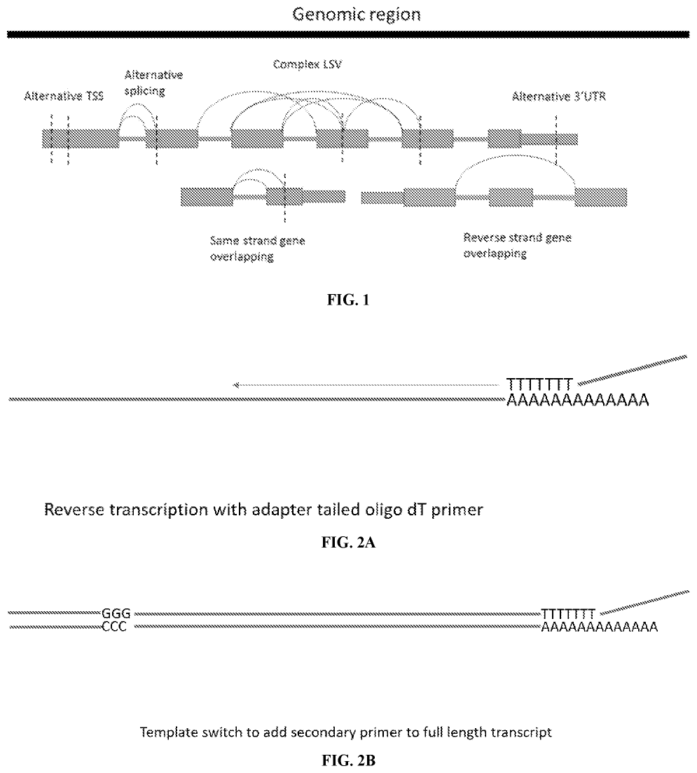

### DETAILED DESCRIPTION

Long read sequencing is one technology currently capable of direct interrogation of gene isoforms. A challenge with performing an analysis of gene isoforms using long read sequencing is the limited read count per sequencer flow cell in combination with a large dynamic range of mRNA gene expression. This can result with data that only includes abundant transcripts. This challenge can be addressed by depleting abundant transcripts with the in vitro use of targeted nucleases, such as CRISPR/CAS. However, to get data for all of the transcripts, it may require sequencing a sample more than once, starting with no of transcripts depletion, then depleting the most abundant transcripts repeatedly until data for all transcripts is obtained. In addition, large guide sets can interfere with the efficiency of depletion. Too many RNPs can compete with each other so that the correct guide sequence ribonucleoprotein (RNP) complex is “blocked” by an RNP with the incorrect guide sequence. Such problems can be solved by reducing the number of guides through serial depletions or slow release depletions.

When only isoform detection without analysis of differential expression is performed, a single sequencer run that can detect all isoforms of all transcripts may be used. In this case full length mRNAs are isolated and balanced to normalize overabundant molecules and to reduce the dynamic range. This approach can be used to interpret the effect of a DNA genome mutation on transcription. In some cases, a candidate DNA mutation is not a protein coding region of a gene and thus difficult to interpret the effect of the mutation. Use of such method provided herein is to determine the impact of a DNA mutation on transcription including, but not limited to, missense mutations, nonsense mutations, and spice donor/acceptor mutations. In some cases, the differential expression of these isoforms is not required.

In an aspect, disclosed herein are methods of detecting an RNA isoform. In some instances, an oligo dT primed reverse transcription reaction is performed on mRNA and template switching is performed with a template switch oligonucleotide (TSO) or with an oligo dT. A first strand and second strand of cDNA molecule can be generated by reverse transcription. In some instances, an adaptor can be attached to a 5′ end and/or a 3′ end of the first or second strand of cDNA molecule can be attached (e.g., ligated, coupled, etc.). In some instances, the adaptor attached cDNA molecule can be further amplified using a primer that binds to the adaptor sequence to produce the amplified products. In some instances, the cDNA molecule is attached with adaptor molecules at its 5′ end and 3′ end (of the same strand) or the 5′ ends of the first and second strands such that two adaptor molecules are located at the opposite sides of the cDNA molecule. In such cases, cDNA molecule can be further amplified using two primers which bind to adaptor sequences. In some instances, two adaptors are identical adaptors. In some instances, two adaptors are distinct adaptors in their sequences.

In some instances, an affinity molecule, (e.g., biotin) can be included on or coupled with the oligonucleotide or primer at the 5′ end or the 3′ end. In some instances, the amplified products coupled with an affinity molecule (e.g., double stranded cDNA molecules coupled with a biotin molecule) can be captured using an agent binding to the affinity molecule (e.g. streptavidin, etc.). Such captured, amplified products can be released from the affinity molecule/capturing agent complex by a nucleic acid modifying moiety (e.g., an endonuclease, a nucleic acid guided endonuclease, CRISPR/CAS, etc.) that can be used to clip off or cleave the amplified products from alternately the 5′ end or the 3′ end. The resulting nucleic acids are enriched for a variety of mRNA isoforms with various 5′ and 3′ ends. A long range sequencing library can be prepared from these enriched nucleic acids. In some cases, the nucleic acid modifying moiety (e.g., the nucleic acid guided endonuclease (e.g., CAS9)) is used to balance the number of molecules for each amplified product (derived from each transcript) that are cleaved by limiting the number of RNPs for each target. This may insure a maximum number of molecules for each transcript. In some cases, each isoform is sequenced to about 4M/22K genes=181× coverage. The 5′ and 3′ cleavage reactions can be done separately to insure collection of all novel transcriptional start sites as well as all alternative polyadenylation sites. In some cases, only 22K guides are needed for each gene at either the 5′ or 3′ end and clean up the data.

In some cases, differential expression analysis is desired. This analysis may be done using removal the top expressed transcripts using negative selection.

In some instances, the nucleic acid modifying moiety comprises Zinc Finger Nucleases (ZFN), Transcription activator like effector nucleases and Clustered Regulatory Interspaced Short palindromic Repeat/Cas based RNA guided DNA nuclease (CRISPR/Cas9), a DNA-guided DNA nuclease, allow for sequence specific degradation of double stranded DNA. These techniques can be used to, for example, cleave a 5′ or a 3′ end of a mRNA transcript.

In some embodiments a moiety that specifically binds to a specific sequence to be cleaved comprises a restriction endonuclease, such as a specific endonuclease that binds and cleaves at a recognition site that is specific to sequence to be cleaved from a 5′ end or a 3′ end of a mRNA transcript.

In some embodiments a population of moieties that specifically bind to a plurality of specific sequences to be cleaved from a 5′ end or a 3′ end of a mRNA transcript comprises at least one restriction endonuclease, two restriction endonucleases or more than two restriction endonucleases.

In some embodiments a moiety that specifically binds to a specific sequence to be cleaved from a 5′ end or a 3′ end of a mRNA transcript comprises a guide RNA molecule. In some embodiments a population of moieties that specifically bind to a specific cleaved from a 5′ end or a 3′ end of a mRNA transcript comprises a population of guide RNA molecules, such as a population of guide molecules that bind to at least one cleaved from a 5′ end or a 3′ end of a mRNA transcript.

A guide RNA molecule comprises sequence that base-pairs with target sequence that is to be cleaved. In some embodiments the base-pairing is complete, while in some embodiments the base pairing is partial or comprises bases that are unpaired along with bases that are paired to non-target sequence.

A guide RNA may comprise a region or regions that form an RNA ‘hairpin’ structure. Such region or regions comprise partially or completely palindromic sequence, such that 5′ and 3′ ends of the region may hybridize to one another to form a double-strand ‘stem’ structure, which in some embodiments is capped by a non-palindromic loop tethering each of the single strands in the double strand loop to one another.

In some embodiments the Guide RNA comprises a stem loop such as a tracrRNA stem loop. A stem loop such as a tracrRNA stem loop may complex with or bind to a nucleic acid endonuclease such as Cas9 DNA endonuclease. Alternately, a stem loop may complex with an endonuclease other than Cas9 or with a nucleic acid modifying enzyme other than an endonuclease, such as a base excision enzyme, a methyltransferase, or an enzyme having other nucleic acid modifying activity that interferes with one or more DNA polymerase enzymes.

The tracrRNA/CRISPR/Endonuclease system was identified as an adaptive immune system in eubacterial and archaeal prokaryotes whereby cells gain resistance to repeated infection by a virus of a known sequence. See, for example, Deltcheva E, Chylinski K, Sharma C M, Gonzales K, Chao Y, Pirzada Z A et al. (2011) “CRISPR RNA maturation by trans-encoded small RNA and host factor RNase III” Nature 471 (7340): 602-7. doi:10.1038/nature09886. PMC 3070239. PMID 21455174; Terns MP, Terns RM (2011) “CRISPR-based adaptive immune systems” Curr Opin Microbiol 14 (3): 321-7. doi:10.1016/j.mib.2011.03.005. PMC 3119747. PMID 21531607; Jinek M, Chylinski K, Fonfara I, Hauer M, Doudna JA, Charpentier E (2012) “A Programmable Dual-RNA-Guided DNA Endonuclease in Adaptive Bacterial Immunity” Science 337 (6096): 816-21. doi:10.1126/science.1225829. PMID 22745249; and Brouns SJ (2012) “A swiss army knife of immunity” Science 337 (6096): 808-9. doi:10.1126/science.1227253. PMID 22904002. The system has been adapted to direct targeted mutagenesis in eukaryotic cells. See, e.g., Wenzhi Jiang, Huanbin Zhou, Honghao Bi, Michael Fromm, Bing Yang, and Donald P. Weeks (2013) “Demonstration of CRISPR/Cas9/sgRNA-mediated targeted gene modification in Arabidopsis, tobacco, sorghum and rice” Nucleic Acids Res. November 2013; 41(20): e188, Published online Aug. 31, 2013. doi: 10.1093/nar/gkt780, and references therein.

As contemplated herein, guide RNA are used in some embodiments to provide sequence specificity to a DNA endonuclease such as a Cas9 endonuclease. In these embodiments a guide RNA comprises a hairpin structure that binds to or is bound by an endonuclease such as Cas9 (other endonucleases are contemplated as alternatives or additions in some embodiments), and a guide RNA further comprises a recognition sequence that binds to or specifically binds to or exclusively binds to a sequence that is to be removed from a sequencing library or a sequencing reaction. The length of the recognition sequence in a guide RNA may vary according to the degree of specificity desired in the sequence elimination process. Short recognition sequences, comprising frequently occurring sequence in the sample or comprising differentially abundant sequence (abundance of AT in an AT-rich genome sample or abundance of GC in a GC-rich genome sample) are likely to identify a relatively large number of sites and therefore to direct frequent nucleic acid modification such as endonuclease activity, base excision, methylation or other activity that interferes with at least one DNA polymerase activity. Long recognition sequences, comprising infrequently occurring sequence in the sample or comprising underrepresented base combinations (abundance of GC in an AT-rich genome sample or abundance of AT in a GC-rich genome sample) are likely to identify a relatively small number of sites and therefore to direct infrequent nucleic acid modification such as endonuclease activity, base excision, methylation or other activity that interferes with at least one DNA polymerase activity. Accordingly, as disclosed herein, in some embodiments one may regulate the frequency of sequence removal from a sequence reaction through modifications to the length or content of the recognition sequence.

Guide RNA may be synthesized through a number of methods consistent with the disclosure herein. Standard synthesis techniques may be used to produce massive quantities of guide RNAs, and/or for highly-repetitive targeted regions, which may require only a few guide RNA molecules to target a multitude of unwanted loci. The double stranded DNA molecules can comprise an RNA site specific binding sequence, a guide RNA sequence for Cas9 protein and a T7 promoter site. In some cases, the double stranded DNA molecules can be less than about 100 bp length. T7 polymerase can be used to create the single stranded RNA molecules, which may include the target RNA sequence and the guide RNA sequence for the Cas9 protein.

Guide RNA sequences may be designed through a number of methods. For example, in some embodiments, non-genic repeat sequences of the human genome are broken up into, for example, 100 bp sliding windows. Double stranded DNA molecules can be synthesized in parallel on a microarray using photolithography.

The windows may vary in size. 30-mer target sequences can be designed with a short trinucleotide protospacer adjacent motif (PAM) sequence of N-G-G flanking the 5′ end of the target design sequence, which in some cases facilitates cleavage. See, among others, Giedrius Gasiunas et al., (2012) “Cas9-crRNA ribonucleoprotein complex mediates specific DNA cleavage for adaptive immunity in bacteria” Proc. Natl. Acad. Sci. USA. September 25, 109(39): E2579-E2586, which is hereby incorporated by reference in its entirety. Redundant sequences can be eliminated and the remaining sequences can be analyzed using a search engine (e.g. BLAST) against the human genome to avoid hybridization against refseq, ENSEMBL and other gene databases to avoid nuclease activity at these sites. The universal Cas9 tracer RNA sequence can be added to the guide RNA target sequence and then flanked by the T7 promoter. The sequences upstream of the T7 promoter site can be synthesized.

Although only about 50% of protein coding genes are estimated to have exons comprising the NGG PAM (photospacer adjacent motif) sequence, multiple strategies are provided herein to increase the percentage of the genome that can be targeted with the Cas9 cutting system. For example, if a PAM sequence is not available in a DNA region, a PAM sequence may be introduced via a combination strategy using a guide RNA coupled with a helper DNA comprising the PAM sequence. The helper DNA can be synthetic and/or single stranded. The PAM sequence in the helper DNA will not be complimentary to the gDNA knockout target in the cDNA library, and may therefore be unbound to the target cDNA library template, but it can be bound to the guide RNA. The guide RNA can be designed to hybridize to both the target sequence and the helper DNA comprising the PAM sequence to form a hybrid DNA:RNA:DNA complex that can be recognized by the Cas9 system.

The PAM sequence may be represented as a single stranded overhang or a hairpin. The hairpin can, in some cases, comprise modified nucleotides that may optionally be degraded. For example, the hairpin can comprise Uracil, which can be degraded by Uracil DNA Glycosylase.

As an alternative to using a DNA comprising a PAM sequence, modified Cas9 proteins without the need of a PAM sequence or modified Cas9 with lower sensitivity to PAM sequences may be used without the need for a helper DNA sequence.

The DNA digestion by Cas9 may be performed before or after cDNA library generation. The Cas9 protein can be added to the DNA (e.g., cDNA library molecules) in vitro (i.e., outside of a cell).

Alternative versions of the assay comprise at least one sequence-specific nuclease, and in some cases a combination of sequence-specific nucleases, such as at least one restriction endonuclease having a recognition site that is abundant in a 5′ or 3′ mRNA sequence region. In some cases an enzyme comprises an activity that yields double-stranded breaks in response to a specific sequence.

Nucleic acid probes (e.g. biotinylated probes) complementary to bait nucleic acids can be hybridized to nucleic acids in solution and pulled down with, e.g., magnetic streptavidin-coated beads. Non bound nucleic acids can be washed away and the captured nucleic acids may then be eluted and amplified for sequencing.

Some embodiments relate to the generation of guide RNA molecules. Guide RNA molecules are in some cases transcribed from DNA templates. A number of RNA polymerases may be used, such as T7 polymerase, RNA Poll, RNA PolII, RNA PolIII, an organellar RNA polymerase, a viral RNA polymerase, or a eubacterial or archaeal polymerase. In some cases the polymerase is T7.

Guide RNA generating templates comprise a promoter, such as a promoter compatible with transcription directed by T7 polymerase, RNA Poll, RNA PolII, RNA PolIII, an organellar RNA polymerase, a viral RNA polymerase, or a eubacterial or archaeal polymerase. In some cases the promoter is a T7 promoter.

Guide RNA templates encode a tag sequence in some cases. A tag sequence binds to a nucleic acid modifying enzyme such as a methylase, base excision enzyme or an endonuclease. In the context of a larger Guide RNA molecule bound to a nontarget site, a tag sequence tethers an enzyme to a nucleic acid nontarget region, directing activity to the nontarget site. An exemplary tethered enzyme is an endonuclease such as Cas9.

In some cases, the stem loop is encoded by a tracr sequence, such as a tracr sequence disclosed in references incorporated herein. Some stem loops bind, for example, Cas9 or other endonuclease.

Guide RNA molecules additionally comprise a recognition sequence. The recognition sequence is completely or incompletely reverse-complementary to a nontarget sequence to be eliminated from a nucleic acid library sequence set. As RNA is able to hybridize using base pair combinations (G:U base pairing, for example) that do not occur in DNA-DNA hybrids, the recognition sequence does not need to be an exact reverse complement of the nontarget sequence to bind. In addition, small perturbations from complete base pairing are tolerated in some cases.

In some cases, the pairing is complete over the length of the recognition sequence. In some cases, the pairing is sufficient for the recognition sequence to tether the tag sequence to the vicinity of a nontarget sequence, or to tether the tag sequence bound to an enzyme to the nontarget sequence.

Guide RNA templates comprise a promoter, a tag sequence and a recognition sequence. In some cases the tag sequence precedes the recognition sequence, while in some cases the recognition sequence precedes the tag sequence. In some cases the construct is bounded by a transcription termination sequence. In some cases, a tag sequence and a recognition sequence are separated by a cloning site, such that a recognition sequence can be cloned into and cloned out of a construct comprising a promoter and a template encoding a tag sequence.

A guide RNA template may be a free molecule or may be cloned in a plasmid, such as a plasmid that directs replication in a cell.

### Definitions

A partial list of relevant definitions is as follows.

“Amplified nucleic acid” or “amplified polynucleotide” as used herein is any nucleic acid or polynucleotide molecule whose amount has been increased at least two fold by any nucleic acid amplification or replication method performed in vitro as compared to its starting amount. For example, an amplified nucleic acid is obtained from a polymerase chain reaction (PCR) which can, in some instances, amplify DNA in an exponential manner (for example, amplification to 2ⁿ copies in n cycles). Amplified nucleic acid can also be obtained from a linear amplification.

“Amplification product” as used herein can refer to a product resulting from an amplification reaction such as a polymerase chain reaction.

An “amplicon” as used herein is a polynucleotide or nucleic acid that is the source and/or product of natural or artificial amplification or replication events.

The term “biological sample” or “sample” as used herein generally refers to a sample or part isolated from a biological entity. The biological sample may show the nature of the whole and examples include, without limitation, bodily fluids, dissociated tumor specimens, cultured cells, and any combination thereof. Biological samples can come from one or more individuals. One or more biological samples can come from the same individual. One non limiting example would be if one sample came from an individual's blood and a second sample came from an individual's tumor biopsy. Examples of biological samples can include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. The samples may include nasopharyngeal wash. Examples of tissue samples of the subject may include but are not limited to, connective tissue, muscle tissue, nervous tissue, epithelial tissue, cartilage, cancerous or tumor sample, or bone. The sample may be provided from a human or animal. The sample may be provided from a mammal, including vertebrates, such as murines, simians, humans, farm animals, sport animals, or pets. The sample may be collected from a living or dead subject. The sample may be collected fresh from a subject or may have undergone some form of pre-processing, storage, or transport.

“Bodily fluid” as used herein generally can describe a fluid or secretion originating from the body of a subject. In some instances, bodily fluids are a mixture of more than one type of bodily fluid mixed together. Some non-limiting examples of bodily fluids are: blood, urine, bone marrow, spinal fluid, pleural fluid, lymphatic fluid, amniotic fluid, ascites, sputum, or a combination thereof.

“Complementary” or “complementarity” as used herein can refer to nucleic acid molecules that are related by base-pairing. Complementary nucleotides are, generally, A and T (or A and U), or C and G (or G and U). Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100%) complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions. Hybridization temperatures are generally at least about 2° C. to about 6° C. lower than melting temperatures (Tₘ).

“Double-stranded” as used herein can refer to two polynucleotide strands that have annealed through complementary base-pairing.

“Library” as used herein can refer to a collection of nucleic acids. A library can contain one or more target fragments. In some instances the target fragments is amplified nucleic acids. In other instances, the target fragments is nucleic acid that is not amplified. A library can contain nucleic acid that has one or more known oligonucleotide sequence(s) added to the 3′ end, the 5′ end or both the 3′ and 5′ end. The library may be prepared so that the fragments can contain a known oligonucleotide sequence that identifies the source of the library (e.g., a molecular identification barcode identifying a patient or DNA source). In some instances, two or more libraries is pooled to create a library pool. Libraries may also be generated with other kits and techniques such as transposon mediated labeling, or “tagmentation” as known in the art. Kits may be commercially available, such as the Illumina NEXTERA kit (Illumina, San Diego, CA).

“Locus specific” or “loci specific” as used herein can refer to one or more loci corresponding to a location in a nucleic acid molecule (e.g., a location within a chromosome or genome). In some instances, a locus is associated with genotype. In some instances loci may be directly isolated and enriched from the sample, e.g., based on hybridization and/or other sequence-based techniques, or they may be selectively amplified using the sample as a template prior to detection of the sequence. In some instances, loci may be selected on the basis of DNA level variation between individuals, based upon specificity for a particular chromosome, based on CG content and/or required amplification conditions of the selected loci, or other characteristics that will be apparent to one skilled in the art upon reading the present disclosure. A locus may also refer to a specific genomic coordinate or location in a genome as denoted by the reference sequence of that genome.

“Long nucleic acid” as used herein can refer to a polynucleotide longer than 1, 2, 3, 4, 5, 6, 7, 8, 9, or 10 kilobases.

“Nucleotide” as used herein can refer to a base-sugar-phosphate combination. Nucleotides are monomeric units of a nucleic acid sequence (e.g., DNA and RNA). The term nucleotide includes naturally and non-naturally occurring ribonucleoside triphosphates ATP, TTP, UTP, CTG, GTP, and ITP, for example and deoxyribonucleoside triphosphates such as dATP, dCTP, dITP, dUTP, dGTP, dTTP, or derivatives thereof. Such derivatives can include, for example, [aS]dATP, 7-deaza-dGTP and 7-deaza-dATP, and, for example, nucleotide derivatives that confer nuclease resistance on the nucleic acid molecule containing them. The term nucleotide as used herein also refers to dideoxyribonucleoside triphosphates (ddNTPs) and their derivatives. Illustrative examples of dideoxyribonucleoside triphosphates include, ddATP, ddCTP, ddGTP, ddITP, ddUTP, ddTTP, for example. Other ddNTPs are contemplated and consistent with the disclosure herein, such as dd (2-6 diamino) purine. In some cases, the nucleotide is a locked nucleic acid. In some cases, the nucleotide is a peptide nucleic acid. In some cases, the nucleotide is an unnatural nucleic acid.

“Polymerase” as used herein can refer to an enzyme that links individual nucleotides together into a strand, using another strand as a template.

“Polymerase chain reaction” or “PCR” as used herein can refer to a technique for replicating a specific piece of selected DNA in vitro, even in the presence of excess non-specific DNA. Primers are added to the selected DNA, where the primers initiate the copying of the selected DNA using nucleotides and, typically, Taq polymerase or the like. By cycling the temperature, the selected DNA is repetitively denatured and copied. A single copy of the selected DNA, even if mixed in with other, random DNA, is amplified to obtain thousands, millions, or billions of replicates. The polymerase chain reaction is used to detect and measure very small amounts of DNA and to create customized pieces of DNA.

The terms “polynucleotides” and “oligonucleotides” as used herein may include but is not limited to various DNA, RNA molecules, derivatives or combination thereof. These may include species such as dNTPs, ddNTPs, 2-methyl NTPs, DNA, RNA, peptide nucleic acids, cDNA, dsDNA, ssDNA, plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA. “Oligonucleotides,” generally, are polynucleoties of a length suitable for use as primers, generally about 6-50 bases but with exceptions, particularly longer, being not uncommon.

A “primer” as used herein generally refers to an oligonucleotide used to prime nucleotide extension, ligation and/or synthesis, such as in the synthesis step of the polymerase chain reaction or in the primer extension techniques used in certain sequencing reactions. A primer may also be used in hybridization techniques as a means to provide complementarity of a locus to a capture oligonucleotide for detection of a specific nucleic acid region. A “primer” may also refer to an oligonucleotide that anneals to a template molecule and provides a 3′ OH group from which template-directed nucleic acid synthesis can occur. Primers comprise unmodified deoxynucleic acids in many cases, but in some cases comprise alternate nucleic acids such as ribonucleic acids or modified nucleic acids such as 2′ methyl ribonucleic acids.

“Primer extension product” as used herein generally refers to the product resulting from a primer extension reaction using a contiguous polynucleotide as a template, and a complementary or partially complementary primer to the contiguous sequence.

“Sequencing,” “sequence determination,” and the like as used herein generally refers to any and all biochemical methods that may be used to determine the order of nucleotide bases in a nucleic acid.

A “sequence” as used herein refers to a series of ordered nucleic acid bases that reflects the relative order of adjacent nucleic acid bases in a nucleic acid molecule, and that can readily be identified specifically though not necessarily uniquely with that nucleic acid molecule. Generally, though not in all cases, a sequence requires a plurality of nucleic acid bases, such as 5 or more bases, to be informative although this number may vary by context. Thus a restriction endonuclease may be referred to as having a ‘sequence’ that it identifies and specifically cleaves even if this sequence is only four bases. A sequence need not ‘uniquely map’ to a fragment of a sample. However, in most cases a sequence must contain sufficient information to be informative as to its molecular source.

As used herein, a library is described as “representative of a sample” if the library comprises an informative sequence of the sample. In some cases an informative sequence comprises about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of a sample sequence. In some cases an informative sequence comprises about 90%, 90%, or greater than 90% of a sample sequence.

The term “biotin,” as used herein, is intended to refer to biotin (5-[(3aS,4S,6aR)-2-oxohexahydro-1H-thieno[3,4-d]imidazol-4-yl]pentanoic acid) and any biotin derivatives and analogs. Such derivatives and analogs are substances which form a complex with the biotin binding pocket of native or modified streptavidin or avidin. Such compounds include, for example, iminobiotin, desthiobiotin and streptavidin affinity peptides, and also include biotin-.epsilon.-N-lysine, biocytin hydrazide, amino or sulfhydryl derivatives of 2-iminobiotin and biotinyl-ε-aminocaproic acid-N-hydroxysuccinimide ester, sulfo-succinimide-iminobiotin, biotinbromoacetylhydrazide, p-diazobenzoyl biocytin, 3-(N-maleimidopropionyl) biocytin. “Streptavidin” can refer to a protein or peptide that can bind to biotin and can include: native egg-white avidin, recombinant avidin, deglycosylated forms of avidin, bacterial streptavidin, recombinant streptavidin, truncated streptavidin, and/or any derivative thereof.

A “subject” as used herein generally refers to an organism that is currently living or an organism that at one time was living or an entity with a genome that can replicate. The methods, kits, and/or compositions of the disclosure is applied to one or more single-celled or multi-cellular subjects, including but not limited to microorganisms such as bacterium and yeast; insects including but not limited to flies, beetles, and bees; plants including but not limited to corn, wheat, seaweed or algae; and animals including, but not limited to: humans; laboratory animals such as mice, rats, monkeys, and chimpanzees; domestic animals such as dogs and cats; agricultural animals such as cows, horses, pigs, sheep, goats; and wild animals such as pandas, lions, tigers, bears, leopards, elephants, zebras, giraffes, gorillas, dolphins, and whales. The methods of this disclosure can also be applied to germs or infectious agents, such as viruses or virus particles or one or more cells that have been infected by one or more viruses.

A “support” as used herein is solid, semisolid, a bead, a surface. The support is mobile in a solution or is immobile.

The term “unique identifier” as used herein may include but is not limited to a molecular bar code, or a percentage of a nucleic acid in a mix, such as dUTP.

The term “about” as used herein in reference to a number refers to that number plus or minus up to 10% of that number. The term used in reference to a range refers to a range having a lower limit as much as 10% below the stated lower limit, and an upper number up to 10% above the stated limit.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Transcriptome Analysis

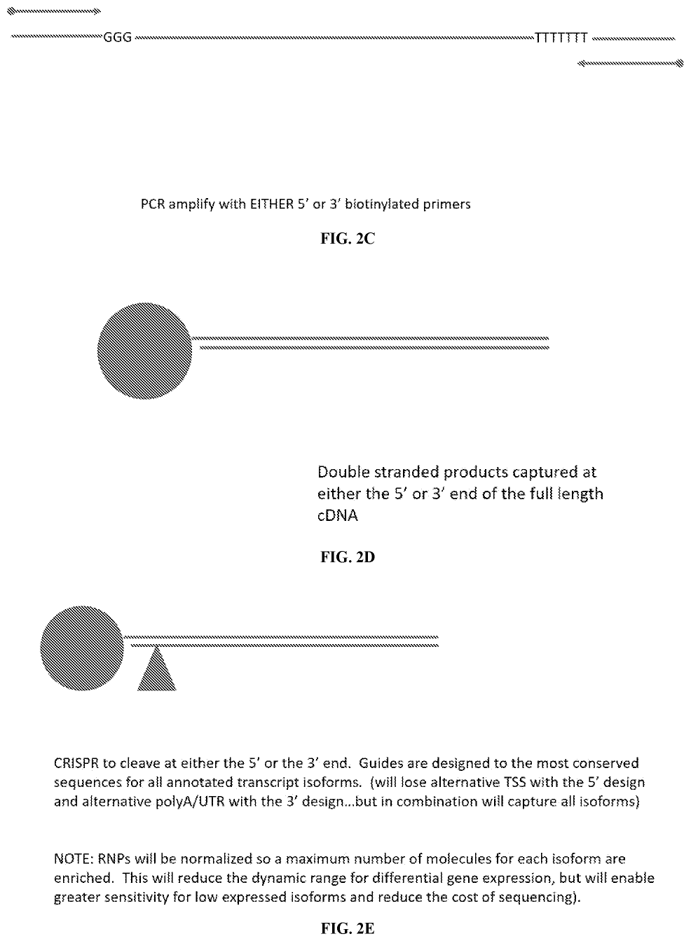

A sample of a tumor is obtained from a patient and analysis of the complete transcriptome of the sample is needed. Messenger RNA (mRNA) is isolated from the tumor sample using standard methods. An adapter tailed oligo (dT) primer is annealed to the isolated mRNA (FIG. 2A). A template switching primer is used to add a secondary primer to the full length transcript (FIG. 2B). The sample is split in two reaction mixtures and each reaction mixture is amplified with primer pairs where either the 5′ primer or the 3′ primer is biotinylated (FIG. 2C). Each reaction mixture is captured at either the 5′ end or the 3′ end using a streptavidin bead (FIG. 2D). CRISPR/CAS9 with a guide designed to cleave at the 5′ end or the 3′ end is added to each reaction mixture. RNPs are normalized so that a maximum number of molecules for each isoform are enriched, enabling greater sensitivity for low expressed isoforms and reduced cost of sequencing (FIG. 2E). The samples are sequenced and information about the transcriptome is obtained.

### Example 2: Transcriptome Analysis

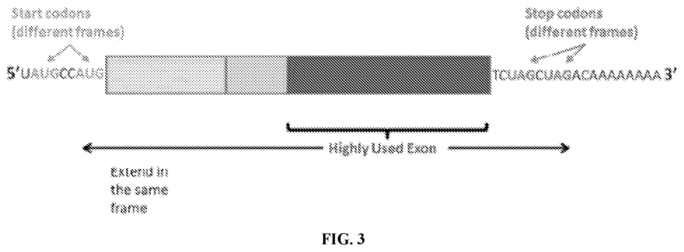

A sample of a tumor is obtained from a patient and analysis of the complete transcriptome of the sample is needed. Messenger RNA (mRNA is isolated from the tumor sample using standard methods. An adapter tailed oligo (dT) primer is annealed to the isolated mRNA (FIG. 2A). A template switching primer is used to add a secondary primer to the full length transcript (FIG. 2B). A cleavage disabled CAS protein complexed with a guide RNA that targets conserved regions of full-length cDNAs is used to capture common transcripts for analysis (FIG. 3).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of detecting an RNA isoform comprising:
(a) contacting a plurality of RNA molecules to a oligo(dT) primer and a reverse transcriptase to produce a plurality of cDNA molecules;
(b) attaching adaptors to a 5′ end and a 3′ end of each of said plurality of cDNA molecules;
(c) amplifying said plurality of cDNA molecules with a first primer and a second primer that bind to said adaptors to produce a plurality of amplification products, wherein said first primer or said second primer comprises a detectable label;
(d) capturing at least a portion of said plurality of amplification products using an agent that binds to said detectable label;
(e) contacting at least a portion of said plurality of amplification products to a guide RNA directed endonuclease and at least one guide RNA that specifically binds to a 5′ end or a 3′ end of at least a subset of said plurality of amplification products, thereby cleaving a 5′ end or a 3′ end of said plurality of amplification products to produce a plurality of cleaved amplification products; and
(f) isolating said plurality of cleaved amplification products, thereby detecting said RNA isoform.

2. The method of claim 1, wherein said attaching of (b) comprises ligating.

3. The method of claim 1, wherein said attaching of (b) occurs concurrently with (a) wherein said oligo(dT) primer comprises said adaptor.

4. The method of any one of claims 1 to 3, wherein said first primer binds to said adaptor at said 5′ end of each of said plurality of cDNA molecules and said second primer binds to said adaptor at said 3′ end of each of said plurality of cDNA molecules.

5. The method of claim 4, wherein said first primer comprises said detectable label and said guide RNA binds to said 5′ end of said subset of said plurality of amplification products.

6. The method of claim 4, wherein said second primer comprises said detectable label and said guide RNA binds to said 3′ end of said subset of said plurality of amplification products.

7. The method of any one of claims 1 to 6, further comprising subsequent to (a) dividing said plurality of cDNA molecules to at least a first reaction volume and a second reaction volume.

8. The method of claim 7, wherein in said first reaction volume said first primer comprises said detectable label and said guide RNA binds to said 5′ end of said plurality of amplification products.

9. The method of claim 7 or claim 8, wherein in said second reaction volume said second primer comprises said detectable label and said guide RNA binds to said 3′ end of said plurality of amplification products.

10. The method of any one of claims 1 to 9, wherein said detectable label comprises biotin.

11. The method of any one of claims 1 to 10, wherein said agent comprises streptavidin.

12. The method of any one of claims 1 to 11, wherein said at least one guide RNA binds specifically to a 5′ end or a 3′ end of said RNA isoform.

13. The method of any one of claims 1 to 12, wherein said at least one guide RNA is provided in an amount relative to an expected amount of said RNA isoform.

14. The method of any one of claims 1 to 13, further comprising sequencing said plurality of cleaved amplification products.

15. A method of detecting an RNA isoform comprising:
(a) contacting a plurality of RNA molecules to a oligo(dT) primer and a reverse transcriptase to produce a plurality of cDNA molecules;
(b) attaching adaptors to a 5′ end and a 3′ end of each of said plurality of cDNA molecules;
(c) amplifying said plurality of cDNA molecules with a first primer and a second primer that bind to said adaptors to produce a plurality of amplification products;
(d) contacting at least a portion of said plurality of amplification products to a cleavage deficient guide RNA directed endonuclease and at least one guide RNA that specifically binds to a 5′ end or a 3′ end of at least a subset of said plurality of amplification products;
(e) capturing at least a portion of said plurality of amplification products using an agent that binds to said cleavage deficient guide RNA directed endonuclease, thereby detecting said RNA isoform.

16. The method of claim 15, wherein said attaching of (b) comprises ligating.

17. The method of claim 15, wherein said attaching of (b) occurs concurrently with (a) wherein said oligo(dT) primer comprises said adaptor.

18. The method of any one of claims 15 to 17, wherein said at least one guide RNA binds specifically to a 5′ end or a 3′ end of said RNA isoform.

19. The method of any one of claims 15 to 17, wherein said at least one guide RNA is provided in an amount relative to an expected amount of said RNA isoform.

20. The method of any one of claims 15 to 19, further comprising sequencing said plurality of captured amplification products.
